(19) United States
(12) Patent Application Publication
(10) Pub. No.: US 2019/0275245 A1
(43) Pub. Date: Sep. 12, 2019

(54) Pressurized-Gas-Driven Infusion Pump and Method of Operating Such an Infusion Pump

(71) Applicant: tricumed Medizintechnik GmbH, Kiel (DE)

(72) Inventor: Karl-Heinz OTTO, Kiel (DE)

(21) Appl. No.: 16/299,562

(22) Filed: Mar. 12, 2019

(30) Foreign Application Priority Data
Mar. 12, 2018 (DE) 10 2018 105 605.2

(51) Int. Cl. A61M 5/168; A61M 5/148

(52) U.S. Cl. CPC A61M 5/16831; A61M 5/1486; A61M 2205/3331; A61M 2005/14513; A61M 2205/18; A61M 2205/581; A61M 2205/3584; A61M 2205/52

(57) ABSTRACT

A pressurized-gas-driven infusion pump having a bladder that forms a medication chamber and a pressure chamber is provided. The pressure chamber acts on the bladder to expel medication through a catheter that is communicatively connected to the medication chamber. A pressure sensor detects the pressure prevailing in the pressure chamber.

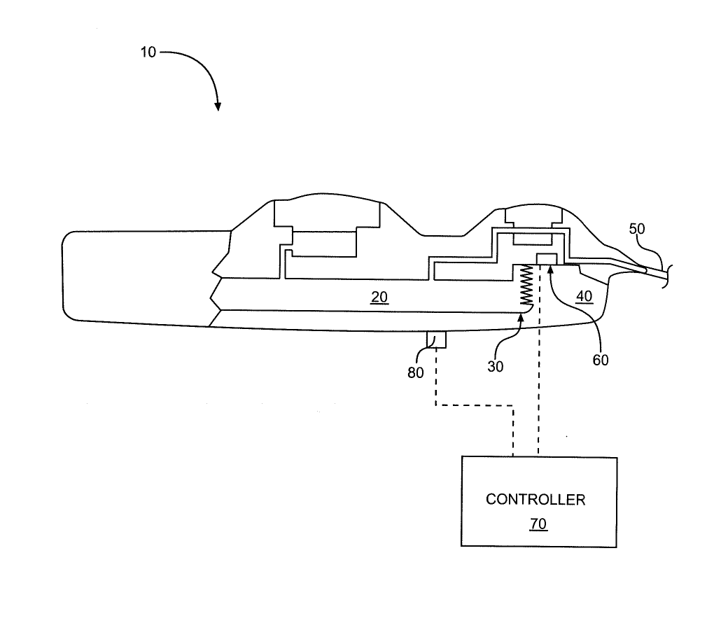

### CROSS REFERENCE TO RELATED APPLICATIONS

This application claims the benefit of German Patent Application No. 10 2018 105 605.2, titled “Pressurized-Gas-Driven Infusion Pump and Method of Operating Such an Infusion Pump” and filed on Mar. 12, 2018, which is incorporated herein by reference.

### TECHNICAL FIELD

The invention relates to a pressurized-gas-driven infusion pump with a bladder forming a medication chamber and a pressure chamber. The pressure chamber acts on the bladder to expel medication through a catheter that is communicatively connected to the medication chamber. The invention also relates to a method for operating such an infusion pump.

### BACKGROUND

Pressurized-gas-driven infusion pumps are well known as implantable infusion pumps. These pumps are made of housings divided into two chambers by a bladder. One chamber, which accommodates a reservoir of a medication, is referred to as a medication chamber. The other chamber, referred to as a pressure chamber, has a drive gas exerting a pressure on the bladder and thus on the medication chamber. The infusion pump is filled with the medication. In use, the drive gas is compressed, so that, due to the expansion of the gas in the pressure chamber, the medication is delivered through a catheter connected to the medication chamber.

The advantage of these known infusion pumps is that the pumps can remain in the body indefinitely and do not require active components that consume energy to perform their function. Therefore, the pumps do not need to be replaced when functioning properly. The disadvantage, however, is that they are difficult to monitor and malfunctions can remain undetected.

For example, the catheter connected to the infusion pump may close due to external mechanical action because of unfavorable positioning or due to deposits in the catheter. Closure of the catheter can result in a reduced delivery, which may not be recognized immediately but only after a longer period of time.

The object of the invention is, therefore, to provide a pressurized-gas-driven infusion pump that can immediately detect malfunctions, especially when there is a shortage of medication.

### SUMMARY OF THE INVENTION

The basic idea of the invention is to detect deviations in pressure changes arising in the pressure chamber due to the expansion of the drive gas from when the medication is dispensed under ideal conditions. The deviations in pressure changes are analyzed to determine a malfunction of the infusion pump. If appropriate, the pump outputs an alarm.

This is accomplished according to the invention by a proposed pressurized-gas-driven infusion pump having a bladder forming a medication chamber and a pressure chamber. The pressure chamber acts on the bladder to expel the medication through a catheter that is communicatively connected to the medication chamber. A pressure sensor is provided that records the pressure prevailing in the pressure chamber.

The pressurized-gas-driven infusion pump preferably has a memory unit connected to the pressure sensor that stores the values detected by the pressure sensor.

In a preferred embodiment, the pressurized-gas-driven infusion pump has a controller connected to the pressure sensor that is configured to output an alarm when the pressure detected by the pressure sensor exceeds or falls below a predetermined pressure and/or a pressure change detected by the pressure sensor exceeds or falls below a predetermined pressure change.

In a particularly preferred embodiment of the invention, the memory unit and/or the controller is configured to wirelessly transmit memory values and/or measured values. The storage of the values makes it possible to read out pressure values stored over the service life of the infusion pump and thereby draw conclusions about the functionality of the infusion pump as well as the quality of the therapy.

Especially advantageous is a pressurized-gas-driven infusion pump that is not only telemetrically readable but also remotely controllable, so that the memory unit and/or the controller are most preferably configured to be programmable via a wireless remote control.

Accordingly, a method for operating a pressurized-gas-driven infusion pump is proposed using a bladder that forms a medication chamber and a pressure chamber. The pressure chamber acts on the bladder to expel medication through a catheter that is communicatively connected to the medication chamber. The method includes the following steps: detecting a pressure prevailing in the pressure chamber and/or detecting a change in pressure in the pressure chamber; and outputting an alarm when the detected pressure exceeds or falls below a predetermined pressure and/or when the detected pressure change exceeds or falls below a predetermined pressure change.

In this process, the detected pressure falling below a predetermined pressure or the detected pressure change falling below a predetermined negative pressure change correlates to a reduced delivery of medication to the patient from the infusion pump. On the other hand, exceeding a predetermined pressure or exceeding a predetermined positive pressure change indicates an increase in a possibly disease-affected body temperature and the associated increased delivery of medication.

The output of the alarm is preferably carried out by emission of an acoustic signal generated by the infusion pump. Alternatively, a telemetric transmission of an alarm can be made that is transmitted to an attending physician, for example, via an appropriate electronic medium, such as the Internet, in particular through a cloud-based system.

In this arrangement, signals indicating a difference between a sensed pressure falling below a predetermined pressure or a sensed pressure change falling below a predetermined negative pressure change and the sensed pressure exceeding a predetermined pressure or the sensed pressure change exceeding a predetermined positive pressure change can also be output. The signals facilitate a simple assignment of the cause of a malfunction and of the respective measures to be taken in response to the malfunction.

It is possible, for example, to integrate the hereby proposed monitoring system in a bolus port of the infusion pump.

In addition, a further pressure sensor, which detects the atmospheric pressure and the pressure acting on the infusion pump due to different body positions of the patient, can be provided in the discharge channel of the medication chamber. The knowledge of these further parameters, which are influenced by the flow rate of the medication in conjunction with the knowledge of the pressure prevailing in the pressure chamber, can be used for mutual compensation and for setting an exact flow rate. This is of particular importance in medications that have high concentrations and are administered at low flow rates.

### BRIEF DESCRIPTION OF THE DRAWING

The Figure shows a schematic sectional view of a particularly preferred design for a pressurized-gas-driven infusion pump according to the invention.

### DETAILED DESCRIPTION OF THE PREFERRED EMBODIMENTS

The invention will be explained in more detail below in reference to an especially preferred exemplary embodiment shown in the Figure.

The Figure shows a schematic sectional view of a particularly preferred design for a pressurized-gas-driven infusion pump 10 according to the invention. The infusion pump 10 has a medication chamber 20 and a pressure chamber 40, which are formed by the housing of the infusion pump 10 and a bladder 30. The pressure chamber 40 is filled with a gas, which acts by expansion on the bladder 30 to expel medication from the medication chamber 20 through a catheter 50. The catheter 50 is communicatively connected to the medication chamber 20 via a throttle section (not labeled). A pressure sensor 60 is provided according to the invention that detects the pressure prevailing in the pressure chamber 40.

Preferably provided is a controller 70 that is connected to the pressure sensor 60 in the infusion pump 10 and is configured to output an alarm when the pressure detected by the pressure sensor 60 exceeds or falls below a predetermined pressure and/or a pressure change detected by the pressure sensor 60 exceeds or falls below a predetermined pressure change.

In addition, a further pressure sensor 80, which detects the atmospheric pressure and the pressure acting on the infusion pump 10 due to different body positions of the patient, can be provided in the discharge channel of the medication chamber 20, for example. The knowledge of these further parameters, which are influenced by the flow rate of the medication in conjunction with the knowledge of the pressure prevailing in the pressure chamber 40, can be used for mutual compensation and for setting an exact flow rate. This is of particular importance in medications that have high concentrations and are administered at low flow rates.

Atmospheric pressure has a large impact on medication delivery by gas-driven infusion pumps. Atmospheric pressure variations correlate with changes in flow rates in implantable infusion pumps that use gas pressure as the driving medium. Certain implantable infusion pumps use n-butane as the driving gas, which produces a pressure of 3.48 bar at a body temperature of 37° C. Hence, atmospheric parameters are an important and inescapable part of the physical environment. Over the course of a day, the air pressure can fluctuate 0.5 to 1 hPa in certain latitudes. However, air pressures between 954.9 and 1060.6 hPa are not uncommon in certain locations (measured at sea level), with a normal air pressure of 1013.25 hPa. Thus, air pressure fluctuations of 105.7 hPa (or 10.43% of the normal air pressure) can be expected. If pressure changes worldwide are considered, air pressure fluctuations of 215.8 hPa (or 21.3%) can be expected. With respect to air pressure changes resulting from altitude changes, the air pressure on a mountain at 2,962 m in altitude can be reduced to 692.8 hPa (or 68.4% of the normal air pressure), for example. Very sensitive pressure sensors should be used to sense these air pressure fluctuations and to set them in relation to the infusion pump pressure of 3.48 bar absolute. This ensures that a flow rate error of +/−1% can be realized.

Also, not depicted in the Figure, but preferably provided, is an antenna for telemetric transmission of the measurement data acquired by the pressure sensor 60 and for receiving control information for the controller 70, e.g., the predetermined pressure values or the predetermined pressure changes, and an acoustic signal generator for outputting the alarm signal.

Finally, an energy storage mechanism (not depicted) for supplying power to the electronic components is also provided.

1. A pressurized-gas-driven infusion pump comprising:
a bladder forming a medication chamber;
a catheter communicatively connected to the medication chamber;
a pressure chamber configured to act on the bladder to cause medication to be expelled through the catheter; and
a pressure sensor configured to sense a prevailing pressure in the pressure chamber.

2. The pressurized-gas-driven infusion pump according to claim 1, further comprising a memory unit connected to the pressure sensor and configured to store values detected by the pressure sensor.

3. The pressurized-gas-driven infusion pump according to claim 2, wherein the memory unit is configured to wirelessly transmit memory values or measured values.

4. The pressurized-gas-driven infusion pump according to claim 2, wherein the memory unit is configured to be programmed wirelessly.

5. The pressurized-gas-driven infusion pump according to claim 1, further comprising a controller connected to the pressure sensor and configured to output an alarm when a pressure detected by the pressure sensor exceeds or falls below a predetermined pressure or when a pressure change detected by the pressure sensor exceeds or falls below a predetermined pressure change.

6. The pressurized-gas-driven infusion pump according to claim 5, wherein the controller is configured to wirelessly transmit memory values or measured values.

7. The pressurized-gas-driven infusion pump according to claim 5, wherein the controller is configured to be programmed wirelessly.

8. A method of operating a pressurized-gas-driven infusion pump including a bladder forming a medication chamber, a catheter communicatively connected to the medication chamber and a pressure chamber configured to act on the bladder to cause medication to be expelled through the catheter, the method comprising:
detecting a pressure prevailing in the pressure chamber; and
outputting a first alarm when the pressure prevailing in the pressure chamber exceeds or falls below a predetermined pressure.

9. The method according to claim 8, wherein outputting the first alarm includes emitting an acoustic signal generated by the infusion pump.

10. The method according to claim 8, further comprising:
detecting a pressure change occurring in the pressure chamber; and
outputting a second alarm when the pressure change exceeds or falls below a predetermined pressure change.

11. A method of operating a pressurized-gas-driven infusion pump including a bladder forming a medication chamber, a catheter communicatively connected to the medication chamber and a pressure chamber configured to act on the bladder to cause medication to be expelled through the catheter, the method comprising:
detecting a pressure change occurring in the pressure chamber; and
outputting a first alarm when the pressure change exceeds a predetermined pressure change.

12. The method according to claim 11, wherein outputting the first alarm includes emitting an acoustic signal generated by the infusion pump.

13. The method according to claim 11, further comprising:
detecting a pressure prevailing in the pressure chamber; and
outputting a second alarm when the pressure prevailing in the pressure chamber exceeds or falls below a predetermined pressure.
